(19) United States
(12) Patent Application Publication
(10) Pub. No.: US 2018/0360732 A1
(43) Pub. Date: Dec. 20, 2018

(54) Oral Care Products and Methods

(71) Applicant: MGE Holdings LLC, Beverly Hills, CA (US)

(72) Inventors: Kourosh Maddahi, Beverly Hills, CA (US); Hessam Nowzari, Beverly Hills, CA (US)

(73) Assignee: MGE Holdings LLC, Beverly Hills, CA (US)

(21) Appl. No.: 16/057,576

(22) Filed: Aug. 7, 2018

Related U.S. Application Data
(63) Continuation-in-part of application No. 15/850,655, filed on Dec. 21, 2017.
(60) Provisional application No. 62/552,650, filed on Aug. 31, 2017.
(60) Provisional application No. 62/465,536, filed on Mar. 1, 2017.
(60) Provisional application No. 62/437,100, filed on Dec. 21, 2016.

(51) Int. Cl. A61K 8/92; A61K 8/20; A61K 8/60; A61K 8/04; A61Q 11/00

(52) U.S. Cl. CPC A61K 8/922; A61K 8/20; A61Q 11/00; A61K 8/042; A61K 8/60

(57) ABSTRACT

Oral care compositions consisting essentially of (i) a sea salt, preferably Dead Sea salt, and (ii) at least two oils selected from the group consisting of lemon peel oil, coconut oil and sage oil, and uses of such compositions to visibly whiten teeth by at least three shades. A delivery system for whitening teeth comprised of a strip formed by combining (i) Dead Sea salt and at least two of coconut oil, lemon peel oil, and/or sage oil and (ii) one or more gelling/thickening agents, wherein the strip is conformable to teeth surfaces and to interstitial spaces between teeth, without permanent deformation thereof, and is removably attached to a release liner.

### CROSS-REFERENCE TO RELATED APPLICATIONS

This application is a continuation-in-part of pending U.S. patent application Ser. No. 15/850,655, filed Dec. 21, 2017. U.S. patent application Ser. No. 15/850,655 claims the benefit of the following three provisional patent applications: (i) U.S. Provisional Application No. 62/437,100, filed Dec. 21, 2016; (ii) U.S. Provisional Application No. 62/465,536, filed Mar. 1, 2017; (iii) U.S. Provisional Application No. 62/552,650 filed Aug. 31, 2017.

### STATEMENT REGARDING FEDERALLY SPONSORED RESEARCH OR DEVELOPMENT

Not applicable.

### FIELD OF INVENTION

The present invention relates to compositions and methods for improving the appearance and health of teeth and gums with oral care products containing naturally-derived ingredients.

### BACKGROUND OF INVENTION

The therapeutic and medicinal benefits of Dead Sea salts have been reported in the scientific literature, typically in connection with diseases of the skin and joints. See, e.g., Uriel Katz et al., “Scientific Evidence of the Therapeutic Effects of Dead Sea Treatments: A Systematic Review,” Seminars in Arthritis and Rheumatism, Vol. 42, No. 2 (October 2012), pp. 186-200, citing Z. Even-Paz, J. Shani, “The dead sea and psoriasis: Historical and geographic background,” Int J Dermatol, Vo. 28, No. 1 (1989), pp. 1-9 (345 g of mineral per liter (34.5% or 34.5 g/100 mL); Id. citing S. Sukenik et al., “The Dead Sea—a unique resort for patients suffering from joint diseases,” Harefuah, Vol. 149, No. 3 (2010), pp. (175-179)(180 to 215 g of mineral per liter). Dan Buskila et al., “Balneotherapy for Fibromyalgia at the Dead Sea,” Rheumatol Int, Vol. 20 (2001), pp. 105-108.

The water of the Dead Sea contains concentrated salts other than NaCl—including, but not limited to, MgCl₂, CaCl₂), KCl, and MgBr₂. Among the separate ions present in the Dead Sea water are chloride (212.4 g/l), magnesium (40.65 g/l), sodium (39.15 g/l), calcium (16.86 g/l), potassium (7.26 g/l), bromide (5.12 g/l), sulfate (0.47 g/l), and bicarbonate (0.22 g/l). See, e.g., I. L. Schamberg, “Treatment of psoriasis at the Dead Sea,” Int J Dermatol, Vol. 17, No. 6 (1978), pp. 524-525; Paz and Shani, supra.

European Patent Application EP1074245A2 discloses use of mineral salt, in particular Dead Sea salts, as an active ingredient in a mouthwash to “assist in combatting bacteria and gum irritation and inflammation”.

Essential oils have been used for the treatment of a variety of ailments since ancient times. The safety and efficacy of essential oils in dentistry have been reported in numerous clinical studies. See, e.g., Namrata Dagli et al., “Essential oils, their therapeutic properties, and implication in dentistry: A Review” J Int Soc Prev Community Dent. Vol. 5, No. 5 (2015), pp. 335-340.

The safety and potential for adverse effects from synthetic ingredients, not only for humans but also the larger ecosystem, have long been of concern. These issues were brought to the forefront by Rachel Carson, in her 1962 book, Silent Spring, which focused on the impact of pesticides, in particular DDT, on birds. A decade later, in 1973, the United States banned DDT. In that same year, manufacturers and producers of health foods and products began organic certification. Two years later, in 1975, Tom's of Maine introduced what it claimed to be the first mass-marketed “natural” toothpaste. The ensuing decades saw an explosive growth in demand for natural and organic products. By 1990, the organic industry had estimated sales of more than $1 billion. In 2006, Tom's of Maine was acquired by the Colgate-Palmolive Company. In 2015, Whole Foods had expanded to 365 stores and reported record revenues of almost $15.5 billion.

While natural personal care products have gained “mainstream” consumer acceptance, concerns remain. Many so-called “natural” products are not “natural”, and contain significant amounts of synthetic ingredients. Other products include “natural” ingredients at de minimis concentrations that do not provide health benefits; instead, natural ingredients are added to these products for purposes of “label copy”.

As access to the internet became more widespread, consumers took steps to publicly question what is natural, posting blogs and comments calling attention to what can be viewed as deceptive or misleading use of the word “natural.” Additionally, the internet has made do-it-yourself personal product recipes (for skincare, haircare, and oral care) available to consumers. See, e.g., http://www.healthyandnaturalworld.com/sage-and-sea-salt-homemade-toothpowder/(¼ cup fresh sage leaves combined with ¾ cup sea salt); see also, http://www.sproutinghealthyhabits.com/homemade-natural-toothpaste/(2 teaspoons of Dead Sea salt; 3 teaspoons of Himalayan pink salt; 2 teaspoons of ground sage; ⅓ cup of stevia powder; 7-8 tablespoons organic unrefined cold pressed coconut oil; 8 drops of tea tree essential oil; 40 drops spear[mint] essential oil; 15 drops of pepper[mint] essential oil; 5 teaspoons of sodium bentonite clay).

Access to a plethora of information on the internet is not, however, without risk. Website content is not subject to review and can be incomplete, inaccurate, or alarmist. Statements that a particular ingredient is “toxic” are often made without proper context. For example, a 1990 report issued by the US National Toxicology Program found “equivocal” evidence that fluoridated drinking water can cause osteosarcoma in male rats. However, exposure to fluoride has been associated with dental and skeletal fluorosis.

Enamel is a quasi-translucent structure which allows the underlying dentin color to show through. Color of teeth (as perceived by others) is determined by the transparency of the enamel, the color tone of the underlying dentin and any imbedded staining contained between the dentin and the enamel surface. It is also associated with the light scattering and absorption properties of the enamel and dentin.

Tooth discoloration (staining) is a multifactorial condition influenced by oral hygiene, diet (foods and beverages that are acidic and/or contain tannins and other chromogenic materials), tobacco usage, and aging. Extrinsic stains appear on the pellicle, the layer of adsorbed salivary proteins and other macromolecules on the dental enamel surface.

These stains can be reduced, and future staining can be prevented, through brushing, rinsing and flossing.

Deeper “intrinsic” staining is caused by long-term accumulation of chromogens within the enamel. Intrinsic staining is typically addressed through professional bleaching procedures (either in-office or at-home). These procedures can include bleaching trays and strips.

The present invention seeks to meet the long-felt but as yet unmet need for natural and naturally derived oral care products (in particular, dentifrices, mouthwashes, whitening strips and whitening gels) that contain safe and effective amounts of natural ingredients useful in cleaning and maintaining healthy, attractive teeth and gums. Products of the present invention are tailored to provide visibly whiter teeth and fresher/cleaner breath.

Improper brushing habits, consuming acidic foods and beverages, using at-home peroxide-containing whitening products, and clenching or grinding teeth all cause teeth sensitivity. These activities wear away and break down the enamel that protects teeth from harsh temperatures and decay. There has been and remains a need for oral care products that visibly whiten teeth without causing, and preferably, reducing teeth sensitivity. Those needs are met by methods of the present invention, in particular methods that use whitening strips consisting essentially of

### BRIEF DESCRIPTION OF THE FIGURES

FIG. 1 illustrates an embodiment of the whitening strip of the present invention [10] attached to a release liner [20]. This embodiment is configured to be applied to the bottom row of teeth.

FIG. 2 illustrates an embodiment of the whitening strip of the present invention [11] attached to a release liner [20]. This embodiment is configured to be applied to the top row of teeth.

FIG. 3 illustrates the VITA TOOTHGUIDE 3-D MASTER® system.

FIG. 4 lists, in table form, shade classifications in the VITA TOOTHGUIDE 3-D MASTER® system and a corresponding Shade Number ranging from 1 to 26.

FIG. 5 illustrates a 7-point plot used by an observer for scoring the chroma and hue of a tooth.

FIGS. 6-10 are tables that present, respectively, improvement (i.e., lightening) in tooth shade by at least 3, 4, 5, 6 and 7 shades, and are based on the TOOTHGUIDE 3-D MASTER® system and corresponding Shade Number (as presented in FIG. 4).

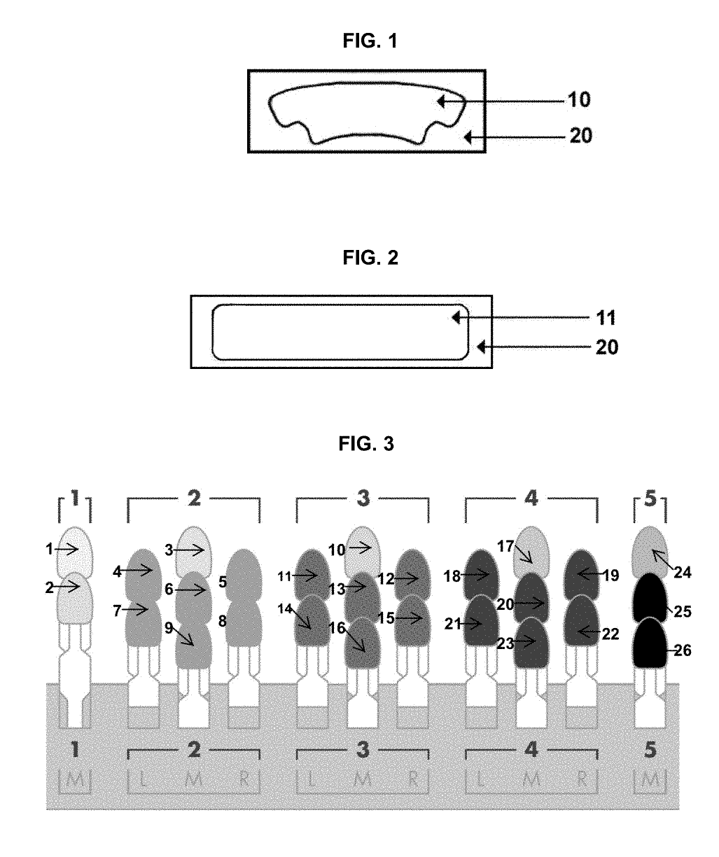

### SUMMARY OF THE INVENTION

The present invention is directed to methods of whitening teeth by administering an oral care product—mouthwash, toothpaste, whitening strip, or whitening gel applied via a pen—consisting essentially of (i) a sea salt, preferably Dead Sea salt, and (ii) a whitening complex consisting of coconut oil, lemon peel oil and sage oil. In toothpaste and mouthwash embodiments of the present invention, the oral care product also contains (xx) xylitol, (yy) aloe vera leaf juice and (zz) at least one essential oil selected from the group consisting of peppermint oil, wintergreen oil, or spearmint oil.

### DETAILED DESCRIPTION OF THE INVENTION

### Definitions

A basic and novel characteristic of the oral care products of the present invention is the absence of: artificial colors, dyes or flavors; paraben or formaldehyde preservatives; sodium lauryl sulfate or sodium laureth sulfate; petroleum-derived glycerin; and genetically modified organisms (CMOs). Collectively, the ingredients listed in this paragraph as absent from the oral care compositions of the present invention are referred to as “Excluded Ingredients.”

In preferred embodiments, a further basic and novel characteristic of the present invention is the ability to whiten without using a peroxide bleaching agent. Non-limiting examples of peroxide bleaching agents include hydrogen peroxide, calcium peroxide, carbamide peroxide, and mixtures thereof. In these preferred embodiments, peroxides are absent from the oral care product and are considered as Excluded Ingredients.

Another basic and novel characteristic of the oral care products of the present invention is non-cytotoxicity within the framework of ISO 10993-5 “Biological Evaluation of Medical Devices—Tests For In Vitro Cytotoxicity,” described in greater detail below.

In mouthwash embodiments, a further basic and novel characteristic of the oral care compositions of the present invention is the absence of ethyl alcohol and/or glycerin; preferably neither ethyl alcohol nor glycerin is present in the mouthwashes of the invention. Certain mouthwash embodiments of the present invention may sometimes be described as “alcohol free.” In labeling personal care and cosmetic products, the term “alcohol,” used by itself, is to be understood by the person having ordinary skill in the art as referring to ethyl alcohol. Accordingly, products labeled as “alcohol free” may contain other alcohols, including menthol or glycerol.

In certain preferred dentifrice embodiments, a further basic and novel characteristic of the oral care compositions of the present invention is the absence of fluoride and/or baking soda; preferably neither fluoride nor baking soda is present in the dentifrices of the invention. In those embodiments, fluoride and/or baking soda are to be considered as Excluded Ingredients. Fluoride may be included in certain formulations within the scope of the invention (e.g., to strengthen tooth enamel and/or remineralize teeth).

Accordingly, the phrase “consisting essentially” used to described oral care compositions of the present invention is to be understood to mean that: (i) the following essential and required component ingredients are present in the oral care compositions of the invention (a) sea salt, preferably Dead Sea salt, and (b) a whitening complex consisting of at least two, and more preferably all three, of coconut oil, lemon peel oil, and sage oil are; and (ii) Excluded Ingredients are not present in the oral care compositions of the invention.

By the term “dentifrice” is meant a preparation for cleansing and polishing the teeth, that may, and preferably does provide one or more therapeutic benefits (as described below). As will be understood by the skilled artisan, a dentifrice (also referred to in the art and in this application as a “toothpaste”) may be formulated as a paste, gel or powder and is preferably applied with a toothbrush.

Dentifrice embodiments of the present invention are administered by dispensing at least about 0.25 grams of a dentifrice of the present invention (commonly described in the scientific literature and in recommendations from dental professionals and toothpaste manufacturers as a “pea sized” amount), and more preferably at least 0.50 grams of dentifrice, and brushing the teeth for at least 30 seconds, preferably for at least 60 seconds, and still more preferably for at least 120 seconds, at least once per day, in the evening prior to going to sleep. In certain preferred embodiments, the dose of toothpaste per administration ranges from about 0.4 to about 0.6 grams.

By “brushing” is meant placing the bristles of a toothbrush in contact with the teeth, preferably at an angle of about 45 degrees to the gum line (where the gums and teeth meet), and moving the bristles in gentle, short strokes along the outer surfaces (cheek side), the inside surfaces (tongue side) and the chewing surfaces of all teeth. The strokes may be in a back-and-forth motion (side-to-side, or up-and-down) or a circular motion. After brushing, the dentifrice is expectorated.

Still more preferably, dentifrice embodiments of the present invention are administered by brushing the teeth for at least 30 seconds, preferably for at least 60 seconds, and still most preferably for at least 120 seconds, twice daily—once in the evening prior to going to sleep; and once in the morning after breakfast.

By the term “mouthwash” is meant a solution that is swished, preferably vigorously, around the mouth, and then expectorated, thereby cleaning the mouth and making the breath smell pleasant.

Mouthwash embodiments of the present invention are administered by swishing about 15-20 ml (about 0.5-0.75 fluid ounces) in the mouth, for a period of time sufficient to contact the teeth, the gums, the roof of the mouth and the tongue. Preferably, mouthwash is swished for at least 30 seconds, preferably for at least 60 seconds, at least once per day, in the evening prior to going to sleep.

Still more preferably, mouthwash embodiments of the present invention are administered for at least 30 seconds, preferably for at least 60 seconds, most preferably for at least 120 seconds, at least once per day twice daily—once in the evening, after brushing prior to going to sleep; and once in the morning after brushing after breakfast.

In especially preferred embodiments, a person practicing an oral care regimen in accordance with present invention (e.g., a consumer or a patient) brushes his/her teeth with a dentifrice of the invention for at least 30 seconds, more preferably for at least 60 seconds, and then swishes a mouthwash of the invention in his/her mouth for a period of at least 30 seconds, preferably at least 60 seconds—a period of time sufficient for the mouthwash to contact the teeth, the gums, the roof of the mouth and the tongue.

Persons practicing methods of the present invention are instructed to abstain from drinking or eating for at least 30 minutes after administering the oral care compositions of the invention; and, since oral care compositions of the invention are not intended for ingestion, to expectorate the oral care composition after use (i.e., brushing in the case of a dentifrice, or swishing in the case of a mouthwash).

As used in the present application the term “dental restoration” is to be understood to mean bridges, crowns, veneers, inlays, and onlays made of porcelain or composite resin.

By the term “whitening strip” is meant a hydratable adhesive film that adheres (sticks) to teeth.

By the term “whitening pen” is meant a gel that is stored in, and administered from, a pen applicator onto the surface of the teeth.

As used in the present application, an “essential oil” is a mixture of terpenic hydrocarbons, especially monoterpenes and sesquiterpenes, and oxygenated derivatives such as aldehydes, ketones, epoxides, alcohols, and esters.

“Non-cytotoxicity” of oral care products of the present invention is confirmed within the framework of ISO 10993-5:2009 based on the widely-used Trypan blue exclusion test. (Trypan blue is a colorant which stains dead cells, i.e., cells with loss of membrane integrity.) More particularly, Balb/c 3T3 clone A31 cells (ATCC CCL 163; 86th passage) are seeded in multi-well plates (24 wells, each 15.5 mm in diameter) at the starting density of 30,000 cells/cm2 in culture medium—Dulbecco's Modified Eagle Medium (DMEM)—supplemented with 10% (v/v) fetal calf serum (FCS). No antibiotics are used. Cultures are incubated at 37° C. in a humidified atmosphere containing 5% (v/v) CO₂, for 24 hours, and are examined with a microscope to verify a sub-confluent monolayer with less than de minimus alteration in morphology. Culture medium is withdrawn and replaced with a solution of one of the following: oral care products of the present invention at 5,000 μg/mL, as well as dilutions 1,500 μg/mL, 500 μg/mL and 150 μg/mL; a “negative” control (phenol, Chemical Abstract Service No. 108-95-2, 0.64 mg/mL); and a “positive” control (DMEM supplemented with 10% (v/v) FCS and 1% antibiotics (v/v). A positive control is defined as statistically significant (30% or greater) inhibition of cell growth as compared to the negative control.

Wells are incubated at 37° C. in a humidified atmosphere containing 5% (v/v) CO₂, for a 24-hour period. Photomicrographs are taken (320× magnification) showing the cell layer in contact with the negative control, the positive control and the oral care product of the present invention. Morphology and cell density of the cultures are observed. At the end of the incubation period, culture medium is removed. Cells are detached for two minutes using 250 μL trypsin (0.05% (w/v) in Hank's balanced solution Ca++ and Mg++ free supplemented with 1 mM EDTA. 250 μL of a Trypan blue solution at 0.2% (w/v) in 0.15 M NaCl and 10% (v/v) FCS are added. Cells are incubated for two minutes. Living cells (uncolored) are counted using a hemocytometer. Cell morphology and cell density of medium treated with 5,000 μg/mL of the oral care product of the present invention are observed to be comparable to those of negative control; neither shows statistically significant (30% or greater) inhibition of cell growth. In contrast, cells treated with the positive control show greater than 50% inhibition on cell growth.

A “consumable chromogenic substance” is a food, beverage or tobacco product that causes tooth staining or discoloration. Non-limiting examples of consumable chromogenic foods include berries, soy sauce and curries. Non-limiting examples of consumable chromogenic beverages include coffee, tea and red wine. Consumable chromogenic tobacco products include cigarettes and smokeless tobacco (that is chewed).

The phrase “at least one” means one or more, and also includes individual components as well as mixtures/combinations.

A “dental arch” is the crescent arrangement of teeth, one on each jaw, that together constitute the dentition.

Numbers used in describing quantities of ingredients and/or reaction conditions are to be understood as being modified in all instances by the term “about.”

Unless otherwise indicated, percentages, parts and ratios are to be understood as based upon the total weight of the composition.

Numerical ranges are meant to include numbers within the recited range, and combinations of subranges between, the given ranges. For example, a range from 1-5, includes 1, 2, 3, 4 and 5, as well as subranges such as 2-5, 3-5, 2-3, 2-4, 1-4, etc.

Component Ingredients

Sea salt is a mixture of inorganic salts from sea water or from inland bodies of salt water. Sea salt may be in the form of a precipitate (on the bottom of a marsh or salt pan or flat) or crystals that float on the surface of the water (known as fleur de sel).

One particularly preferred sea salt suitable for use in the oral care products of the present invention is Dead Sea salt, which is a mixture of natural hygroscopic minerals and micronutrients found in the Dead Sea and is comprised of sodium chloride, magnesium, potassium, and calcium chlorides and bromides. A non-limiting compositional analysis of Dead Sea salt versus common salt is presented in the table below:

| | Dead Sea Salt (%) | Common Salt (%) |
|---|---|---|
| H₂O | 37.5 | 0.33 |
| MgCl₂ | 32.2 | 0.18 |
| KCl | 24.5 | 0.14 |
| NaCl | 5.6 | 99.2 |
| CaCl₂ | 6.23 | 0.15 |
| Br⁻ | 0.35 | 0.052 |
| Rb⁺ | 0.025 | — |
| Li⁺ | 1.0 | — |
| Fe³⁺ | 0.00203 | 0.00016 |
| Al³⁺ | 0.00037 | 0.000028 |
| SO₄²⁻ | 0.00916 | 0.0311 |
| Sr²⁺ | 0.00153 | 0.00047 |
| Mn²⁺ | 0.00023 | 0.0038 |

S. Halevy et al., J. Eur. Acad. Dermatol. Venereol., Vol. 9, pp. 237-242 (1997).

Another preferred sea salt suitable for use in the oral care products of the present invention is Himalayan salt, which is harvested from the Punjab Region of Pakistan, and is comprised of sodium chloride (about 95-98%), with about 2 to 3% polyhalite (potassium, calcium, magnesium, sulfur, oxygen, hydrogen), fluoride, iodine, and smaller amounts of other trace minerals.

Dead Sea salt is present in oral care products of the invention of the invention at a concentration of less than about 3%, preferably a concentration of from about 0.1% to 2%, still more preferably a concentration of from about 0.5% to about 1.5%.

Citrus limon (lemon) peel oil is the volatile oil obtained from the peel of Citrus limon.

Salvia officinalis (sage) oil is the essential oil derived from the herbal plant, Salvia officinalis.

Cocos nucifera (coconut) oil is an oil obtained from the kernel or seed of Cocos nucifera.

In certain preferred embodiments, whitening oral care products of the present invention contain coconut oil and lemon peel oil in a ratio of about 1:1.

In other preferred embodiments, whitening oral care products of the present invention contain coconut oil, lemon peel oil, and sage oil in a ratio of about 1:1:1.

Xylitol is the pentahydric alcohol.

Aloe vera leaf juice useful in the present invention preferably contains (i) glycosides at a concentration of at least about 1%, preferably at least about 2%, and still more preferably at about 3%, as well as (ii) at least two, preferably three, anti-inflammatory agents selected from the group of anthraquinones, sterols, auxins and gibberellins and (iii) and immunomodulatory muccopolysachharides, preferably Acemannan.

Spearmint oil is the volatile oil obtained from the leaves of Mentha viridis (also known as Mentha spicata).

Peppermint oil is a volatile oil obtained from the whole plant Mentha piperita.

Wintergreen oil is the volatile oil obtained from the leaves of Gaultheria procumbens.

In certain embodiments, oral care products of the present invention contain one or more essential oils selected from the group consisting of spearmint oil, wintergreen oil, and peppermint oil.

In one even more preferred embodiment, oral care products of the present invention include peppermint oil and one of wintergreen oil or spearmint oil.

In another even more preferred embodiment, oral care products of the present invention include wintergreen oil and one of peppermint oil or spearmint oil.

In a still further even more preferred embodiment, oral care products of the present invention include spearmint oil and one of peppermint oil or wintergreen oil.

In especially preferred embodiments, the oral care products of the present invention contain spearmint oil, peppermint oil, and wintergreen oil.

Menthol, an alcohol that can be isolated from peppermint or other mint oils, can also be used in oral care products of the present invention.

In certain embodiments, oral care products of the present invention also preferably include oils of one or both of Ocimum basilicum (basil) or Eugenia caryophyllus (clove flower).

In certain preferred embodiments of the invention, basil oil is present at a concentration of up to about 0.5%.

Other essential oils that may be included in oral care products of the present invention include Melaleuca alternifolia (tea tree) leaf oil, the oil distilled from the leaves of the Melaleuca alternifolia, and Zingiber officinale (ginger) root oil, which is obtained from the dried rhizomes of Zingiber officinale.

Dentifrice embodiments of the present invention may include mild abrasives (to remove debris and residual surface stains), humectants (to prevent water loss in the toothpaste), thickening products, also known in the art as binders (to stabilize the toothpaste formula), flavoring products (for taste) and detergents (to create foaming action).

Mild abrasives suitable for use in the toothpaste embodiments of the present invention include calcium carbonate, dehydrated silica gels, hydrated aluminum oxides, magnesium carbonate, phosphate salts and silicates. Silica, also called silicone dioxide, bentonite clay and hydrated silica are minerals. Some toothpastes of the present invention preferably contain hydrated silica.

Humectants that may be, and preferably are, ingredients in toothpastes of the present invention include glycerin, preferably vegetable glycerine, propylene glycol, and sorbitol.

Glycerin, a sugar alcohol that can be synthesized or obtained from natural sources, is an especially preferred humectant used in toothpastes of the invention. Preferably, vegetable glycerin is used in oral care compositions of the present invention.

Non-limiting examples of thickening products that may be, and preferably are included in toothpaste embodiments of the present invention include gums and colloids. Preferred colloids are of marine origin, even more preferably seaweeds.

Two preferred gums are xanthan gum and biosaccharide gum-1; both are polysaccharides derived from the fermentation of carbohydrates. Xanthan gum is derived from glucose or corn syrup. Biosaccharide gum-1 is derived from sorbitol.

Carrageenan, a polysaccharide hydrocolloid obtained from edible red seaweeds in the Gigartinaceae or Solieriaceae families, may be, and preferably is, present in toothpastes of the invention.

### EXAMPLES

The following examples illustrate compositions and methods of practicing of the present invention in some of its embodiments; the examples should not be construed as limiting the scope of the invention. Other embodiments will be apparent to one skilled in the art from consideration of the specification and examples. It is intended that the specification, including the examples, is considered exemplary only without limiting the scope and spirit of the invention.

Some of the examples illustrate preferred embodiments of the invention. Variations of these preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, unless otherwise indicated herein or otherwise clearly contradicted by context, the inventions include all modifications and equivalents of the subject matter disclosed and recited in the claims appended hereto as permitted by applicable law.

Whitening Dentifrice of the Invention

Whitening toothpastes of the present invention contain the following ingredients at the following concentrations.

Dead sea salt is present in whitening toothpastes of the present invention at a concentration of from about 0.5% to about 3.0%, preferably from about 0.75% to about 1.75%, and most preferably from about 1.0% to about 1.5%.

In whitening toothpastes of present invention in which coconut oil is a component of the whitening complex, coconut oil is present at a concentration of from about 0.001% to about 5%, preferably from about 0.005% to about 1%, and most preferably from about 0.09% to about 0.9%.

In whitening toothpastes of the present invention in which lemon peel oil is a component of the whitening complex, lemon peel oil is present at a concentration of from about 0.001% to about 5%, preferably from about 0.005% to about 1%, and most preferably from about 0.09% to about 0.9%.

In whitening toothpastes of the present invention in which sage oil is a component of the whitening complex, sage oil is present at a concentration of from about 0.001% to about 5%, preferably from about 0.005% to about 1%, and most preferably from about 0.09% to about 0.9%.

Aloe vera leaf juice is preferably a component of the whitening toothpastes of the present invention at a concentration of from about 10% to about 90%, preferably from about 20% to about 85%, and most preferably from about 40% to about 70%.

Xylitol is preferably a component of the whitening toothpastes of the present invention at a dose of about 0.1 gram/brushing. The concentration of xylitol can range from about 5% to about 30%, preferably from about 15% to about 25%, and most preferably from about 17% to about 22%.

Peppermint oil, when present in whitening toothpastes of the present invention, is included at a concentration of from about 0.001% to about 0.07%, preferably from about 0.005% to about 0.06%, and most preferably from about 0.01% to about 0.03%.

Spearmint oil, when present in whitening toothpastes of the present invention, is included at a concentration of from about 0.01% to about 2%, preferably from about 0.05% to about 1.5%, and most preferably from about 0.09% to about 1.2%.

Wintergreen oil, when present in whitening toothpastes of the present invention, is included at a concentration of from about 0.05% to about 1%, preferably from about 0.1% to about 0.5%, and most preferably from about 0.2% to about 0.4%.

Basil oil, when present in whitening toothpastes of the present invention, is included at a concentration of from about 0.001% to about 0.05%, preferably from about 0.005% to about 0.03%, and most preferably from about 0.02% to about 0.035%.

Clove flower oil, when present in whitening toothpastes of the present invention, is included at a concentration of from about 0.001% to about 0.05%, preferably from about 0.005% to about 0.04, and most preferably from about 0.01% to about 0.03%.

Vegetable glycerin is preferably present in whitening toothpastes of the present invention at a concentration of from about 5% to about 20%, preferably from about 8% to about 12%, and most preferably about 10%.

Carrageenan is preferably present in whitening toothpastes of the present invention at a concentration of from about 0.05% to about 5%, preferably from about 0.1% to about 0.3%, and most preferably from about 0.15% to about 0.25%.

Xanthan gum is preferably present in whitening toothpastes of the present invention at a concentration of from about 0.1% to about 1.0%, preferably from about 0.4% to about 0.8%, and most preferably from about 0.5% to about 0.7%.

Titanium dioxide may be used in whitening toothpastes of the present invention at a concentration of from about 0.01% to about 1.0%, preferably from about 0.03% to about 0.07%, and most preferably from about 0.4% to about 0.6%.

Hydrated silica may be used in whitening toothpastes of the present invention at a concentration of from about 5% to about 25%, preferably from about 10% to about 20%, and most preferably from about 14% to about 17%.

Sodium methyl cocoyl taurate is preferably used in whitening toothpastes of the present invention at a concentration of from about 0.3% to about 2%, preferably from about 0.5% to about 1.5%, and most preferably from about 0.8% to about 1.2%.

Whitening Mouthwash of the Invention

Whitening mouthwashes of the present invention contain the following ingredients at the following concentrations.

Dead Sea salt is present in whitening mouthwashes of the present invention at a concentration of from about 0.5% to about 5%, preferably from about 0.75% to about 3%, and most preferably from about 1% to about 2%.

In whitening mouthwashes of the present invention in which coconut oil is a component of the whitening complex, coconut oil is present at a concentration of from about 0.001% to about 5%, preferably from about 0.1% to about 0.7%, and most preferably from about 0.15% to about 0.6%.

In whitening mouthwashes of the present invention in which lemon peel oil is a component of the whitening complex, lemon peel oil is present at a concentration of from about 0.001% to about 5%, preferably from about 0.1% to about 0.7%, and most preferably from about 0.15% to about 0.6%.

In whitening mouthwashes of the present invention in which sage oil is a component of the whitening complex, sage oil is present at a concentration of from about 0.001% to about 0.09%, preferably from about 0.005% to about 0.05%, and most preferably from about 0.01% to about 0.03%.

Aloe vera leaf juice is preferably present in whitening mouthwashes of the present invention at a concentration of from about 10% to about 90%, preferably from about 20% to about 85%, and most preferably from about 50% to about 70%.

Xylitol is preferably present in whitening mouthwashes of the present invention at a concentration of from about 5% to about 30%, preferably from about 7% to about 15%, and most preferably from about 8% to about 12%.

Basil oil, when present in whitening mouthwashes of the present invention, is included at a concentration of from about 0.005% to about 0.5%, preferably from about 0.01% to about 0.2%, and most preferably from about 0.02% to about 0.1%.

Clove flower oil, when present in whitening mouthwashes of the present invention, is present at a concentration of from about 0.005% to about 0.075%, preferably from about 0.01% to about 0.04%, and most preferably from about 0.01% to about 0.03%.

Peppermint oil, when present in whitening mouthwashes of the present invention, is included at a concentration of from about 0.005% to about 0.12%, preferably from about 0.01% to about 0.1%, and most preferably from about 0.02% to about 0.09%.

Spearmint oil, when present in whitening mouthwashes of the present invention, is included at a concentration of from about 0.01% to about 1%, preferably from about 0.02% to about 0.17%, and most preferably from about 0.05% to about 0.15%.

Wintergreen oil, when present in whitening mouthwashes of the present invention, is included at a concentration of from about 0.03% to about 1%, preferably from about 0.05% to about 0.5%, and most preferably from about 0.1% to about 0.45%.

Toothpastes and mouthwashes of the present invention may also, optionally, in place of wintergreen oil, peppermint oil and/or spearmint oil, include flavorants and/or taste masking products known in the art materials. The flavorants may be of natural or synthetic origin. Non-limiting examples of such flavorants include but are not limited to fruit flavors such as cherry, strawberry, lemon, lime, orange, watermelon, or a mixture of fruit flavors (sometimes known as tutti frutti).

Whitening Strip

Whitening strips of the present invention (also referred to in the examples below as “Test Strips”) consist essentially of (i) Whitening Composition in which (a) sea salt, preferably Dead Sea salt, and (b) a Whitening Complex consisting of two, and preferably three, of coconut oil, lemon peel oil and/or sage oil are added to a Gel Carrier and (ii) a two-sided flexible strip that is (a) safe for contact with the structures of the oral cavity of humans (i.e., non-toxic, non-corrosive, and non-irritating), (b) conformable to a plurality of adjacent teeth surfaces and to interstitial spaces between teeth, without permanent deformation thereof.

The Gel Carrier provides adhesive attachment sufficient to hold a strip in place for a desired period of time to allow the Active Ingredient(s) to be delivered to, and act on, the teeth surface, and thereby provide a desired clinical outcome—namely, whitening of teeth by at least two shades, preferably at least three shades, more preferably at least four shades, even more preferably at least five shades, still more preferably at least six shades; and, in certain embodiments, by at least seven shades. See discussion below for methods of measuring whitening (i.e., lightening) of teeth by at least two shades.

The Gel Carrier is comprised of at least one gelling agent known in the art and (a) is generally recognized to be safe for oral use, (b) does not readily dissolve in saliva, and (c) does not react with, inactivate, or cause the degradation or loss of desired function of Active Ingredients in the Whitening Composition.

Non-limiting examples of suitable gelling agents that may, and preferably are, components of the Gel Carrier, include: carboxypolymethylene, carboxymethyl cellulose, carboxypropyl cellulose, poloxamer, carrageenan, magnesium aluminum silicate (commercially-available under the tradename Veegum® from Vanderbilt Materials, LLC), carboxyvinyl polymers (including Carbomer), and natural gums such as gum karaya, xanthan gum, guar gum, gum arabic, gum tragacanth, and mixtures thereof.

Carbomers are a class of homopolymers of acrylic acid crosslinked with an allyl ether of pentaerythritol, an allyl ether of sucrose, or an allyl ether of propylene. One preferred Carbomer gelling agent is carboxypolymethylene, available from B. F. Goodrich Company under the tradename Carbopol®. Particularly preferred Carbopols include Carbopol® 934, 940, 941, 956 and mixtures thereof. Especially preferred is Carbopol® 956.

Another preferred gelling agent is polyvinylpyrrolidone. In certain embodiments polyvinylpyrrolidone is of a molecular weight in a range of 1,000,000 to 1,500,000. In certain embodiments, polyvinylpyrrolidone has molecular weight of about 50,000 to about 300,000.

Additional gelling agents that are suitable for use in the whitening strips of the present invention include celluloses (hydroxy ethyl or propyl cellulose, ethyl cellulose) and Polyox® water-soluble resins available from Dow Corning (Midland, Mich.).

Polyox® resins are nonionic, high molecular weight water-soluble poly (ethylene oxide) polymers with molecular weights ranging from 100,000 to about 8,000,000.

Gantrez® copolymers—monoalkyl esters of poly (methyl vinyl ether/maleic acid) with varying ester groups—available from Ashland are also suitable for to thicken the whitening compositions and promote adhesion between the strip and teeth surfaces.

Gelling agents as described above is/are preferably present in the Whitening Composition at a concentration from about 0.1% to about 15%, preferably from about 1% to about 10%, more preferably from about 2% to about 8%, and most preferably from about 4% to about 7%, by weight.

The viscosity and tackiness of the Whitening Composition(s) are such that the whitening strip (a) remains adhesively attached to a plurality of adjacent teeth for the desired contact time without substantial slippage under friction from the lips, tongue, and other soft tissue rubbing against the strip (e.g., during mouth movements associated with talking) and (b) can be easily removed by the wearer by applying a peel force using a finger or fingernail, and not requiring the use of an instrument, a chemical solvent, or undue friction.

In preferred embodiments, the peel force required to remove the strip is from about 1 gram to about 50 grams for a strip that is 1.5 cm in width. Even more preferably, for a strip that is 1.5 cm in width, the peel force is from about 5 grams to about 40 grams, and more preferably from about 10 grams to about 30 grams.

Preferably, the Whitening Composition has a viscosity of from about 200 to about 1,000,000 at low shear rates. More preferably, the viscosity is from about 100,000 to about 800,000 cps, and even more preferably the viscosity is from about 400,000 to about 600,000 cps.

While the Whitening Composition(s) is preferably in the form of a gel, it can also be in the form of a liquid or paste that meets the above viscosity and tackiness profile.

The Whitening Composition is applied to a first side of the flexible strip; a release liner (defined below) is placed on top of the Whitening Composition. The first side is applied to the teeth surface after the release liner is removed.

The time required for the Active Ingredients to be delivered to and act upon the teeth surface, and thereby provide a desired clinical outcome (whitening) can range from several minutes to several hours. Accordingly, a second side of the strip (opposite the side covered by the release liner) is designed to serve as a protective barrier to minimize, and preferably substantially prevent, the Whitening Composition from coming into contact with saliva as well as the wearer's lips, tongue, and other soft tissue surrounding the teeth (e.g., papilla, marginal gingiva, gingival sulcus, inter dental gingiva, gingival gum structure on lingual and buccal surfaces up to and including muco-gingival junction and the pallet).

In preferred embodiments of the present invention the whitening strip is applied to the teeth for a period of time of at least about 5 minutes, preferably at least about 10 minutes, more preferably for at least about 15 minutes, even more preferably for at least about 20 minutes, most preferably for at least about 30 minutes; but not for longer than about 60 minutes.

The flexible strip component of the whitening strip may be a single layer of material or a laminate, or a plurality or layers of materials or laminates, a heterogeneous mixture of ingredients, separate stripes or spots or other patterns of ingredients, or a combination of laminates, layers, stripes, and/or spots of ingredients. The one or more materials/laminates and other components/ingredients (i) meet the flexural rigidity characteristics described below.

The flexible strip may be constructed of polymers, natural and synthetic woven materials, non-woven materials, foil, paper, rubber, and combinations thereof.

In preferred embodiments, the material(s) used to construct the flexible strip is/are polymers that are substantially water impermeable and/or polymers or gelling agents (described above) that are hydratable. Suitable polymers include, but are not limited to, polyethylene, ethylvinylacetate, ethylvinyl alcohol, polyethylene, polyesters (such as Mylar®, manufactured by DuPont), fluoroplastics (such as Teflon®, manufactured by DuPont), and combinations thereof.

In certain embodiments, the whitening strip of the invention is less than about 2 mm thick, preferably less than about 1 mm thick, and more preferably less than about 0.5 mm thick, and even more preferably less than about 0.1 mm thick.

Preferably, the whitening strip of the invention is configured in a shape that has rounded corners, by which is meant not having any sharp angles or points.

The size (length and width) of the whitening strip of the invention will vary based upon one or more factors, including the number of teeth to be treated (i.e., whitened), the size of the teeth, and personal preference of the wearer.

Preferably, the whitening strip of the invention is of a length that covers at least the front 6-10 teeth of the upper or lower rows of teeth that are visible when the wearer is smiling. Optionally, the strip covers the entire upper and/or lower rows of teeth.

In certain embodiments, the length of the whitening strip of the invention is from about 2 cm to about 12 cm, preferably from about 4 cm to about 9 cm.

The width of the whitening strip of the invention will vary depending on whether the strip is intended to cover the front surface of the teeth, or wrap around the teeth and cover both surfaces of the teeth.

In certain embodiments, the width of the whitening strip of the invention is from about 0.5 cm to about 4 cm, preferably from about 1 to about 2 cm.

The flexural stiffness of the whitening strip of the invention is determined by strip thickness, strip width, and material modulus of elasticity. One method for measuring flexural stiffness of a polymeric strip is ASTM D2912-95, which employs a strain gauge wired to a microammeter. In accordance with this method, the rigidity of the strip is read directly from the microammeter and expressed as grams per centimeter of strip width. A non-limiting, but preferred, instrument for measuring flexural stiffness is Handle-O-Meter®, available from Thwing-Albert Instrument Co. of Philadelphia, Pa.

In certain embodiments, the whitening strip of the invention has a flexural stiffness of less than about 5 grams/cm, preferably a flexural stiffness less than about 4 grams/cm, more preferably less than about 3 grams/cm, and most preferably from about 0.1 grams/cm to about 1 grams/cm.

In other embodiments, the flexural stiffness of the whitening strip of the invention is substantially constant and does not significantly change during normal use. For example, in these embodiments, the strip does not need to be hydrated for the strip to achieve flexural stiffness in the above-specified ranges.

Low flexural stiffness (in the above-specified ranges) is an important property of the whitening strip of the invention, which allows the strip to drape over, and conform to, the contoured surfaces of teeth of the wearer's mouth, including gaps between adjacent teeth. By designing strips to have a flexural stiffness within the above-specified ranges, there are minimal residual forces within the strip that would cause it to return to an original (i.e. starting) configuration which is substantially flat.

Additionally, the flexibility (low flexural stiffness) of the whitening strip of the invention enables the strip to contact soft tissue over an extended period of time without physical irritation.

The whitening strip of the invention may, in certain embodiments, be configured to have shallow recesses to create reservoirs (depots) for the whitening composition of the Invention and/or different oral care products. These recesses may be filled, for example, with: Dead Sea salt and/or coconut oil; a peroxide (e.g., hydrogen peroxide or carbamide peroxide); a combination of one or both of Dead Sea salt and/or coconut oil and a peroxide (for example, Dead Sea salt and a peroxide; coconut oil and a peroxide; Dead Sea salt, coconut oil and a peroxide).

The release liner may be formed from any material which exhibits less affinity for the Whitening Composition than the Whitening Composition exhibits for (a) itself and (b) for the flexible strip. The release liner preferably comprises a rigid sheet of material such as polyethylene, paper, polyester, or other material which is then coated with a non-stick type material. The release liner material may be coated with wax, silicone, polyester such as Teflon®, fluoropolymers, or other non-stick type materials. The release liner material may crack when the strip is flexed.

The release liner may be cut to substantially the same size and shape as the strip or the release liner may be cut larger than the strip to provide a means for separating the release liner from the strip.

The release liner may have a tab, or be scored, to facilitate separation from the strip. Alternatively, the release liner may be two overlapping pieces in a bandage-type design.

In preferred embodiments, the Whitening Composition(s) is homogeneous, and is preferably coated onto the strip in a uniform manner.

In alternate embodiments, more than one Whitening Composition(s) may be coated onto the strip in a series of separate stripes or spots or other patterns of ingredients.

While preferred embodiments of the present invention are directed to whitening oral care products that contain as the only Active Ingredients a sea salt and a Whitening Complex, some embodiments may also contain as a further Active Ingredient an additional ingredient that whitens the surface of teeth (i.e., bleaching or stain removal), including, but not limited to, peroxides, metal chlorites, perborates, percarbonates, peroxyacids, and combinations thereof.

The peroxide compound should provide an amount of hydrogen peroxide equivalent of from about from about 0.1% to about 40%, preferably from about 0.5% to about 10%, and most preferably from about 1% to about 7%, by weight of the substance. Preferred peroxide compounds include hydrogen peroxide, calcium peroxide, carbamide peroxide, and mixtures thereof.

A particularly preferred peroxide is carbamide peroxide. To deliver the desired amount of hydrogen peroxide equivalent carbamide peroxide would be included in the whitening composition in an amount of from about 0.1% to about 45% and preferably from about 3% to about 20% on a weight/weight basis.

Preferred metal chlorites include calcium chlorite, barium chlorite, magnesium chlorite, lithium chlorite, sodium chlorite, and potassium chlorite.

Other active ingredients suitable for inclusion in the strips of the present invention for purposes of whitening the surface of teeth include hypochlorite and chlorine dioxide.

In one preferred embodiment, whitening strips of the present invention include Dead Sea salt, coconut oil, lemon peel oil and sage oil, each a concentration of up to about 10.0%, preferably each a concentration of up to about 5.0%, and even more preferably at a concentration of at least about 3.0%.

Each of these ingredients is preferably present at a concentration of up to about 800 grams, and more preferably up to about 400 grams.

In one especially preferred embodiment, whitening strips of the present invention include Dead Sea salt, coconut oil, lemon peel oil and sage oil in a ratio of about 1:1:1:1.

In one especially preferred embodiment, the whitening strips of the present invention preferably include a hydratable adhesive matrix comprised of glycerol, carbomer and a cellulose, preferably carboxymethylcellulose.

Whitening Gel (Applied Via Pen Applicator)

Certain embodiments of the present invention are directed to methods for visibly whitening teeth (and removing stains) and/or preventing or reducing the amount of staining by using a Whitening Pen in which a gel composition is stored in, and dispensed from, a pen application. The gel composition is preferably the Whitening Composition that is used in the whitening strips of the present invention.

Assessment of Tooth Whitening

Improvement in tooth shade (i.e., lightening) achieved by practicing the methods of the present invention can be determined by visually assessing the tooth before, during and after treatment in accordance with the present invention by choosing the closest shade from a dental shade guide that matches the color of the tooth.

Shade can be, and in preferred embodiments of the present invention, is described according to the Munsell color space in terms of value, chroma, and hue, each defined below.

“Value” indicates the lightness of a color. Lightness is defined by the Commission Internationale de l'Eclairage (“CIE”) as the “brightness of an area judged relative to the brightness of a similarly illuminated area that appears to be white or highly transmitting.”

“Chrome” is the degree of color saturation, and is defined by the CIE as the “colourfulness of an area judged as a proportion of the brightness of a similarly illuminated area that appears white or highly transmitting.”

“Hue” is the attribute of a color that enables the clinician to distinguish between different families of color, and defined as the “attribute of a visual perception according to which an area appears to be similar to one of the colours: red, yellow, green, and blue, or to a combination of adjacent pairs of these colours considered in a closed ring”. Hue is represented by A, B, C or D on the commonly used VITA Classic shade guide.

In certain embodiments, improvement in tooth shade (i.e., lightening) is measured using the VITA TOOTHGUIDE 3-D MASTER® system—a dental shade guide comprised of 26 shade tabs based on a color classification principle where the values of lightness, chroma, and hue have been positioned an equal distance from each other. See FIG. 3.

The VITA TOOTHGUIDE 3-D MASTER® system is organized into five primary value levels, arranged from the lightest (value level 1) to the darkest (value level 5), left to right. These values, in turn, are further distributed (subdivided) based on chroma and hue. Within each of the five groups, shade tabs are arranged vertically according to chroma (pale to saturated and horizontally according to hue (yellowish to reddish).

US Patent Application Pre-Grant Publication 2009/0233253 describes the VITA TOOTHGUIDE 3-D MASTER® system as follows: Shades are assigned to linear groups numbered 1 through 5 denoting different value levels. Level 1 value is higher (closer to white). As the group number increases toward 5, value is lower (closer to black). In groups 2, 3, and 4, shade tabs are linearly arranged by hue. “L” group tabs, on the left side of each value grouping, are more prominent in the yellow range of color. “R” group tabs, arranged on the right side of each value grouping, are more prominent in the red range of color. “M” group tabs, arranged in the middle of each value grouping, are in-between in hue with a balance of yellow and red. All tabs in each value group are also arranged vertically, one behind the other. Tabs in the M group are further designated 1, 2, and 3. Tabs in the L and R groups are further designated 1.5 and 2.5.

In embodiments of the present invention in which improvement in tooth shade is assessed based on the VITA TOOTHGUIDE 3-D MASTER® system, value is determined first, followed by chroma. Hue is determined last, by matching with shade tabs of the value and chroma already determined. In these embodiments, the phrase “improvement in shade” is assessed by a three-step process as described immediately below.

Using a light source with a color temperature corresponding to “daylight light” (from 4600K-6500K, more preferably from 5500K to 6500K), lightness, chroma and hue are determined.

First, the lightness of a patient's tooth is determined by holding the shade guide (i.e., sample) directly in front of the tooth, at a distance of approximately 25-30 cm. The trained observer (e.g., clinician) assigns a “value” (lightness level) on a six-point scale of 0, 1, 2, 3, 4 or 5, with 0 being light and 5 being dark. When selecting the lightness level, the observer begins by looking at the dark end (5) and moves toward the light end (0).

Next, the observer matches chroma (pale or saturated). The observer starts by removing a shade guide with the middle hue (M) from the selected lightness level. The observer then chooses one of three chroma within the selected lightness level.

As a final step, the observer determines hue by comparing the selected shade guide to the shade guide directly to the left (L) and right (R), and determines whether the tooth is more yellow (L) or red (R) than the middle (M) shade guide.

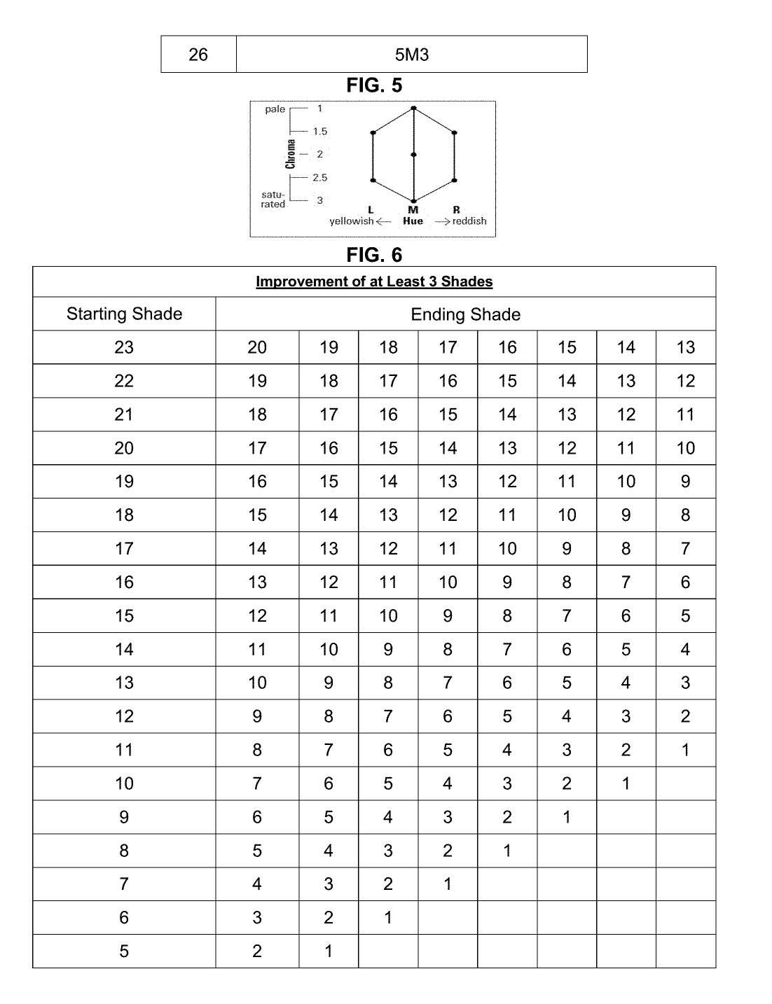

The observer may, and in certain embodiments does, score chroma and hue using the 7-point plot shown in FIG. 5.

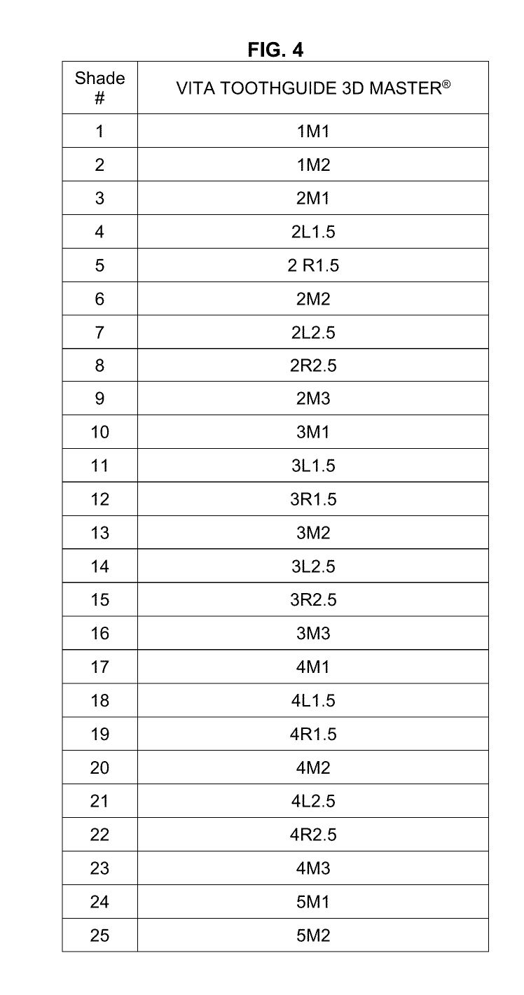

In preferred embodiments of the present invention the trained observer assigns an initial (baseline) shade based on the VITA TOOTHGUIDE 3-D MASTER® system, and then assigns a corresponding Shade Number ranging from 1 to 26. See FIG. 4. The observer then tracks improvement (lightening) of tooth shade based on reduction (lowering) of the shade number.

In particularly preferred embodiments, practicing the methods of the present invention produces an improvement in tooth shade by lowering shade number by at least three.

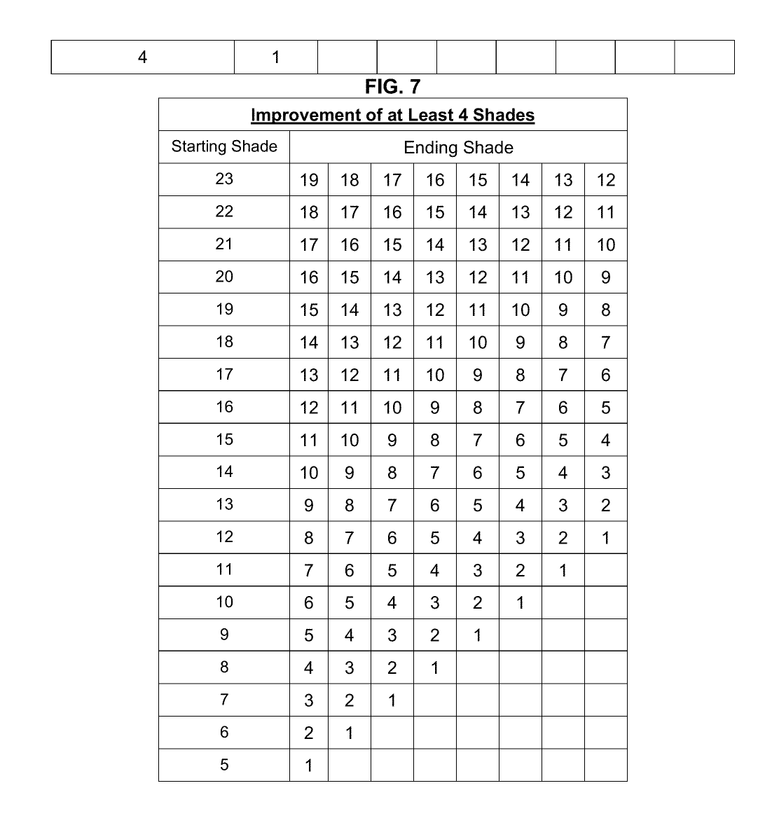

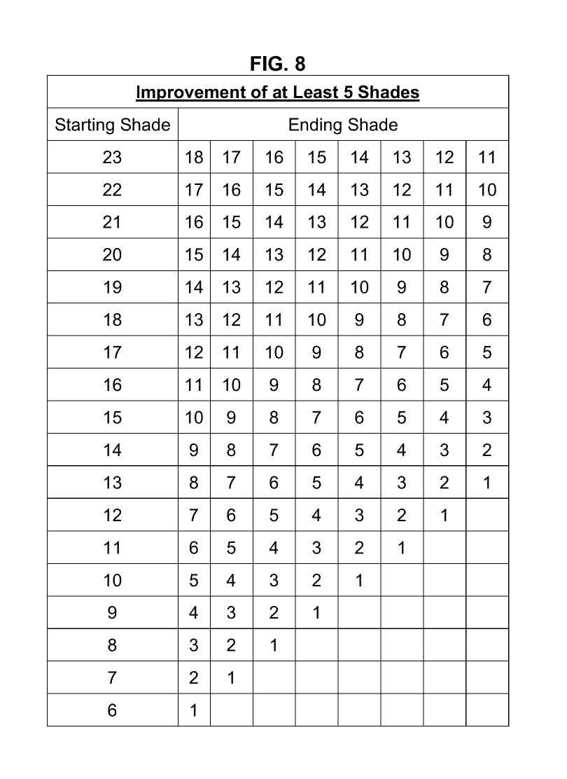

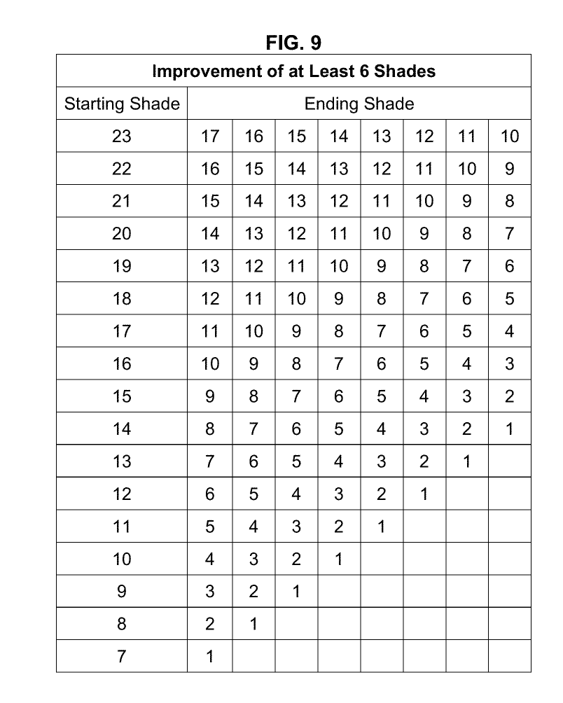

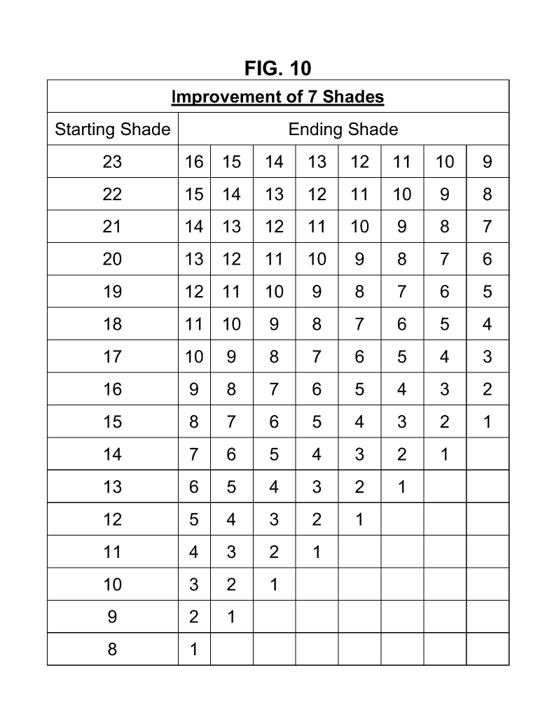

The following are non-limiting examples of what is meant by the phrase “improvement in tooth shade by lowering shade number by at least three”. A consumer starts with a shade number of “15” (3R2.5) and after administering one or a combination of oral care products of the present invention (e.g., a whitening dentifrice, and a whitening mouthwash; or a whitening dentifrice, a whitening mouthwash, and a whitening strip) the consumer's tooth shade number becomes “12” (3R1.5) or “11” (3L1.5) or “10” (3M1), preferably “9” (2M3), more preferably “8” (2R2.5) or “7” (2L2.5) or “6” (2M2), and even more preferably “5” (2R1.5) or “4” (2L1.5) or “3” (2M1). In the above example, a change from “15” to “12” represents an improvement of three (3) shade numbers, an improvement from “15” to “11” represents an improvement of four (4) shade numbers, an improvement from “15” to “10” represents an improvement of five (5) shade numbers, etc. Improvements of three, four, five, six, and seven shades lighter are summarized in FIGS. 6-10.

The VITA TOOTHGUIDE 3-D MASTER® is further described in the following scientific and patent publications, which are incorporated, in pertinent part, by reference: AJ Hasell et al., “Determination of VITA Classical shades with the 3D-Master shade guide,” Acta Odontol. Scand. Vol. 71, No. 3-4, pp. 721-726 (2014); N. Corcordel, et al, “The linear shade guide design of Vita 3D-master performs as well as the original design of the Vita 3D-master,” J. Oral Rehabil., Vol. 37, pp. 860-865 (2010); US Patent Application Pre-Grant Publication 2010/005563.

Other tooth shade guides are known to the person having ordinary skill in the dental arts and can be used to assess lightening achieved by practicing the methods of the present invention. Non-limiting examples of shade guide systems include: VITA Classical (VITA North America, Yorba Linda, Calif.) and CHROMASCOP® (Ivoclar Vivadent, Inc., Amherst, N.Y.).

The VITA CLASSICAL A1-D4® system is arranged by four hues—“A”, “B”, “C”, or “D”, and subdivided as follows: A1, A2, A3, A4—reddish to brownish; B1, B2, B3, B4—reddish to yellowish; C1, C2, C3, C4—greyish; D2, D3, D4—reddish to greyish.

The CHROMASCOP® system is arranged by value levels covering the area of the CIE 1976 (L*a*b*) color space of “natural teeth”. Five groups—ranging from white, yellow, light brown, grey, to dark brown—have tabs representing different chroma and hue.

In the CIE 1976 (L*a*b*) system, “L*” value represents comparative lightness/darkness (lower L* being indicative of darker), and “a*” and “b*” values represent chromaticity coordinates (red-green and blue-yellow, respectively). See A. R. Robertson, “The CIE 1976 color-difference formulae,” Color Res. Appl. Vol. 2, pp. 7-11 (1977). Each of the L*, a* and b* can be plotted in three-dimensional space to characterize a color in absolute terms. The magnitude of the difference between two colors, as perceived by the human eye, is proportional to the distance between two points defining the two colors on the three-dimensional plot. The difference between the two colors, the Euclidean distance (ΔE), is defined by the following equation: ΔE*ab=[(ΔL*)²+(Δa*)²+(Δb*)²]^{1/2}. An increase in b* indicates tooth color is trending towards yellow; a decrease in b* means the color is trending towards blue.

Clinical Testing Examples

The examples that follow illustrate the practice of the present invention in some of its embodiments, but should not be construed as limiting the scope of the invention. Other embodiments will be apparent to one skilled in the art from consideration of the specification and examples. It is intended that the specification, including the examples, is considered exemplary only without limiting the scope and spirit of the invention.

Some of the examples illustrate preferred embodiments of the invention. Variations of these preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, unless otherwise indicated herein or otherwise clearly contradicted by context, the inventions include all modifications and equivalents of the subject matter disclosed and recited in the claims appended hereto as permitted by applicable law.

Twenty previously-extracted (20) teeth, classified as healthy by an experienced dentist using a loupe and headlamp, were selected and sterilized. By “healthy” is meant the teeth did not have any dental restorations or exhibit structural damage (e.g., pitting, enamel decay).

Two chips (i.e., a pair) were created from each tooth. Each chip had an approximate size of 5 mm×5 mm×3 mm in size. An impermeable plastic was affixed to the cut surfaces of the chips. Each pair was stained in a separate vial by immersion in a concentrated black tea solution “overnight” (for about 12 hours). The concentrated black tea solution was produced by steeping 2 teabags (LIPTON'S° yellow label black tea) in ½ cup of boiling water for 30 minutes. After staining, two (2) groups—Group 1 and Group 2—were created. Each group consisted of ten pairs—A and B.

Chips in Group 1 were used to compare whitening of stained dental enamel by (i) a whitening mouthwash of the invention (“Test Mouthwash”) versus (ii) a commercial whitening mouthwash, CREST® 3D Whitening Mouthwash (“Prior Art Mouthwash”). As detailed below, Test Mouthwash achieved a similar degree of whitening as the Prior Art Mouthwash (no statistical difference, p>0.05).

Chips in Group 2 were used to compare whitening of stained dental enamel by (i) a whitening strip of the invention (“Test Strips”) versus (ii) a commercial whitening strip, CREST® 3D Whitening Strips (“Prior Art Strips”). As detailed below, Test Strips achieved a similar degree of whitening as Prior Art Strips (no statistical difference, p>0.05).

Effect of Whitening Strips of the Invention on Dental Enamel

Chips in Group 1A were treated with Test Strips. Chips in Group 1B were treated with Prior Art Strips. By “treated” is meant a whitening strip was applied to the chip for 60 minutes; thereafter, the chips were immersed in a saliva solution for approximately one (1) minute. The saliva solution was collected “fresh”, without stimulation, and was filtered to remove particulate matter. The above procedure was repeated a total of ten (10) times.

At baseline (after staining but prior to the first treatment), and after each treatment (as defined below), L* and b* values were recorded using a reflectance spectrophotometer colorimeter (PCE-CSM 1, PCE Instrumentation, Alicante, Spain). Chips were photographed individually under standardized lighting conditions at each study time point.

Treatment with the Test Strip whitened the enamel surface of the chips, but had no visible effect on the underlying dentin. Mean standard deviation of L* was 9%. With the exception of T=8 hours (when b* for Test Strips was higher, and therefore, better than Prior Art Strips) differences in b* were not statistically significantly (p>0.05). (Because the difference at T=8 hours barely exceeded the mean standard deviation, this data point was viewed as a minor outlier.)

Effect of Whitening Mouthwash of the Invention on Dental Enamel

Chips in Group 2A were treated with a Test Mouthwash. Chips in Group 2B were treated with Prior Art Mouthwash. By “treated” is meant immersion for about one minute in either the Test Mouthwash or the Prior Art Mouthwash; thereafter, the chips were immersed in a fresh saliva solution as described above for approximately one (1) minute. The above procedure was repeated a total of ten (10) times. Each treatment (i.e., immersion) with a mouthwash is viewed as the clinical equivalent of use for thirty (30) consecutive days at the recommended dose administered (rinsed) twice daily, each administration having a duration of 1 minute.

Colorimetry and clinical photographs were taken at the following time points: baseline (T=0), and the following seven time points post-immersion: 1 hour; 2 hours; 3 hours; 6 hours; 12 hours; 24 hours; 48 hours. Chips were photographed individually under standardized lighting conditions at each study time point.

Baseline b* values for the 2 groups did not differ significantly (p>0.05). Except as noted below, there were no statistically significant differences (p>0.05) between the effects of treatment with the Test and Prior Art Mouthwash formulations. At 6 hours, chips treated with the Prior Art Mouthwash showed a somewhat greater shift to a yellow hue. At 48 hours, chips treated with the Test Mouthwash showed a somewhat more yellow hue.

Baseline L* values for Groups 2A and 2B did not differ significantly (p>0.05). The efficacy of the Test Mouthwash and the Prior Art Mouthwash in terms of lightening did not differ significantly over the first three hours (p>0.05). The maximum effectiveness of the Test Mouthwash was observed after about 1 hour of treatment. Increased treatment time was observed to add approximately 6% of additional lightening effect.

Effect of Whitening Strips of the Invention on Dental Restorations

Dental restorations from fourteen (14) different manufacturers/suppliers—six (6) constructed from porcelain and eight (8) from composite materials—were used to prepare chips having an approximate dimension 3 mm×2 mm×1 mm. 30 chips from the same supplier were mounted on three handling strips, each handling strip consisting of ten chips. All chips were stained by immersion in a concentrated black tea solution produced by steeping 2 teabags (LIPTON's® yellow label black tea) in ½ cup of boiling water for 2 hours.

One handling strip served as a control, and was stored in water at temperature of 4 C and 100% humidity, and protected from ambient light in a sealed and labeled double-walled container. The remaining two handling strips (also referred to below as “active intervention”) underwent “treatment”, i.e., application of either a Test Strip or a Prior Art Strip to the chips for 60 minutes and, thereafter, immersion in a saliva solution (as described above in Example 1) for approximately one (1) minute. Prior to treatment, the active intervention handling strips were stored in a 0.5% chloramine-T solution for 48 hours prior to start of the study.

At baseline (time=0) and the following seven (7) specific time points, each measured from baseline, the surface structure of each sample was examined by light microscopy [Olympus® Cap Optical SZX Light Microscope (Center Valley, Pa.), at 35× magnification] to determine changes in the chip surface, and specifically to identify cracking, pitting, dissolution, roughening or erosion: 1 hour, 3 hours, 6 hours, 9 hours, 12 hours, 18 hours, 24 hours. No visible surface damage was observed in any of the samples.

In addition, the color of each chip was measured by reflectance spectrophotometer (PCE-CSM 1, PCE Instrumentation (Alicante, Spain) D-65 standard illuminant; 4 mm diameter measuring area) and photographed under standard lighting conditions (i.e., the same lighting conditions were used throughout the study).

Color changes (e.g., lightening) were observed in both the Test Strips and Prior Art Strips, with no statistically significant difference between the groups; there was no difference between the control and the test groups at any time point, based on two-way ANOVA followed by Bonferroni post-test P>0.05.

Effect of Whitening Strip of Invention on Surface of Porcelain and Composite Materials

Surface changes in dental restorations after use of Test Strips and Prior Art Strips were examined by Light Microscopy, Scanning Electron Microscopy (SEM), and Profilometry.

126 chips from the control and treatment groups were examined for surface damage—cracks, pits or fissures—under light microscopy (Olympus® Cap Optical SZX Light Microscope) at three different magnifications: 10×, 25× and 35×. No surface damage was observed in either the treatment groups (when compared to the controls) regardless of color, material and exposure duration.

Approximately half of the active intervention chips, and an equal amount of control chips, were affixed to Aluminum specimen mount disks using colloidal silver liquid (Ted Pella, Inc., Redding, Calif.) and coated with 9.31 nm thick gold alloy. Samples were photographed using a Scanning Electron Microscope (SEM) [FEI Magellan 400L FE-SEM, Nanolab Technologies, Inc. (Milpitas, Calif.)] at three (3) magnifications (500×, 1000× and 3000×).

Chips in treatment and control groups were compared to determine the extent of microstructural surface (if any) by a blinded evaluator with experience and training in evaluating enamel and dental restorations using SEM. The chips were scored as “mild”, “moderate” or “severe”. Mild: small, individual, non-connected minor pitting or roughening covering less than 10% of the sample surface. Moderate: individual or connected pitting or roughening covering less than 25% of the sample surface. Severe: widespread pitting or roughness covering more than 25% of the sample surface. At magnifications of from 500× to 3,000×, greater surface roughening was consistently observed in composite and porcelain chips treated with Prior Art Strips than those treated with Test Strips.

The remaining half of the active intervention chips, and an equal amount of control chips, were examined by profilometry (Perthometer M2, Mahr GmbH; Göttingen, Germany) with the following specifications and calibration: profilometer tip diameter=2.4 mm; accuracy=0.5 mm/sec; measuring path=5.5 mm; cutoff value=250 μm). Five measurements were taken for each chip sample. Average surface roughness (based on five measurements) was recorded and evaluated by repeated measures of variance technique (α=0.05); coincidences were eliminated by Duncan test. After every seventh specimen (total of 35 measurements), the profilometer was recalibrated. Based on profilometry, significant increases in post-whitening roughness (ANOVA, P>0.05) were observed in composite chips treated with Prior Art Strips, but not in composite chips treated with Test Strips. No significant increases in post-whitening roughness (ANOVA, P>0.05) were observed in porcelain chips treated with Test Strips and Prior Art Strips.

Tooth Shade Lightening Effects of Whitening Strip of Invention

Five extracted teeth samples (numbered 1-5) with matching degrees of staining were selected. Tooth color was measured by an experienced, blinded, trained observer using standard colorimetry and the VITA TOOTHGUIDE 3D MASTER® tooth shade grading system. Lighting was indirect and standardized and all measurements were repeated three times.

At baseline, samples 1-3 had a shade of 3M2; samples 4 and 5 had a baseline shade of 2M1. Mean L* was 69.66; mean b* was 13.35. A whitening strip of the invention was administered to each sample for ten hours—representative of 20 treatments, each lasting 30 minutes. Samples 1-3 lightened to shade 1M1. Samples 4 and 5 also lightened to shade 1M1. After treatment, mean L* was 79.84; mean b* was 11.85.

Tooth Shade Lightening Effects of Whitening Mouthwash of Invention

Five extracted teeth samples (numbered 1-5) with matching degrees of staining were selected. Tooth color was measured by an experienced, blinded, trained observer using standard colorimetry and the VITA TOOTHGUIDE 3D MASTER® tooth shade grading system. Lighting was indirect and standardized and all measurements were repeated three times.

At baseline, samples 1-3 had a shade of 4M1; samples 4 and 5 had a baseline shade of 3M2. Mean L* was 68.99; mean b* was 11.17. Samples were treated by immersion in a whitening mouthwash of the invention for 48 hours. Samples 1-3 lightened to shade 3M1. Samples 4 and 5 also lightened to shade 3M1. After treatment, mean L* was 71.30; mean b* was 10.10.

In Vivo Effects of Whitening Strip of Invention on Tooth Sensitivity and Gingival Irritation

Ten participants ages 18-45 (mean 29.4 years)—each with (i) a minimum of 16 clinically and radiographically healthy teeth, as determined by clinical examination and (ii) an absence of any pathological symptoms, gingival recession or tooth sensitivity prior to enrollment—were recruited to participate in a 10-day, randomized, double-blinded, clinical study.

At the commencement of the study, each subject received forty (40) identically packaged, coded whitening strips—20 Test Strips (as defined above) and 20 Prior Art Strips (as defined above)—as well as a new soft-bristled Oral B° toothbrush and CREST Total Care® toothpaste. Subjects brushed their teeth twice daily and used only the oral hygiene products as described above. For example, no mouthwash or floss was used. Subjects applied four whitening strips—one on each designated quadrant of the mouth (i.e., upper left, lower left, upper right, lower right)—to the outer surface of the teeth for thirty (30) minutes each day. After treatment, subjects rinsed their mouths with water. After each application, on a daily basis, subjects completed a questionnaire with respect to (a) tooth sensitivity and (b) gingival irritation. More particularly, subjects self-reported sensitivity using a 5-point Likert Scale and gingival irritation as “yes” or “no”). On Days 5 and 10 of the study, a blinded clinician evaluated (a) gingival irritation by visual inspection on a scale ranging from 0 to 3 and (b) sensitivity after a 3-second blast of pressurized air—(yes or no). Lower levels of gingival irritation were reported in the group that used Test Strips. Comparable levels of sensitivity were reported in users of the Test Strips and the Prior Art Strips, both as self-reported by questionnaire and as evaluated by clinical evaluation at on Days 5 and 10.

Stain Prevention by Whitening Pen of Invention

The effects of a whitening gel of the invention (Whitening Composition in Gel Carrier) administered by a pen applicator on enamel stain development during timed exposure to coffee and red wine were studied. As detailed below, in comparison to uncoated teeth, application (i.e., coating) teeth with the whitening gel of the present invention prevented enamel staining (darkening as measured in terms of changes in L*) for an additional 10 minutes of coffee exposure and an additional 20 minutes of red wine exposure.

Test Sample Preparation:

20 extracted teeth that appeared healthy to the naked eye and inspection under the light microscope (×10) were bisected into 20 pairs. Standardized photography and colorimetry measurements were taken at baseline. One-half of each tooth was treated with two coats of the whitening gel of the present invention; the remaining half tooth was uncoated. On the “treatment” side, a first coat was administered (e.g., painted). After drying for 30 seconds, a second coat was applied. Sample pairs were immersed in individual 20 mL aliquots of freshly brewed VIA® INSTANT coffee (Starbucks Corp., Seattle, Wash.) according to the instructions on the packet (i.e., about one 3.3-gram packet add to 8 fluid ounces of hot water). The remaining 10 sample pairs were immersed in individual 20 mL aliquots of red wine (CHARLES SHAW® Cabernet Sauvignon, Trader Joe's Company, Monrovia, Calif.). Standardized photography and colorimetry to determine L* and b* values were repeated five times—every 10 minutes, for a total of 50 minutes.

Coffee Staining:

Coffee exposure of non-coated and coated teeth exhibited significant decrease in the L* value (darkening) over time. Teeth coated with whitening gel of the present invention began to exhibit staining after 30 minutes of immersion in coffee. In contrast, non-coated teeth began to exhibit staining after 20 minutes of coffee immersion. Changes in L* values were assessed by one-way ANOVA, as well as ANOVA with post hoc Tukey test. Minimal and not significant staining was observed at 10-minute and 20-minute time points (P>0.01). Compared to baseline, L* increased significantly for non-coated teeth at 20-, 30-, 40-, and 50-minute time points; respectively: P<0.01, P<0.01, P<0.001, P<0.001. For teeth coated with whitening gel of the present invention, significant L* increases (versus baseline) were observed at 30-, 40-, and 50-minute time points, but these changes were less significant than non-treated teeth; respectively, P<0.05, P<0.01, P<0.01.

Red Wine Staining:

Immersion of non-coated teeth resulted in a significant decrease of L* values after each of five time points (i.e, 10 minutes, 20 minutes, 30 minutes, 40 minutes, 50 minutes; respectively: P<0.01, P<0.01, P<0.01, P<0.001, P<0.001; one-way ANOVA followed by Tukey post-test). Teeth coated with whitening gel of the present invention began to darken significantly only after being immersed in red wine for 30 minutes, with progressive darkening at each subsequent time point. Using one-way ANOVA followed by Tukey post-test, L* values were assessed at 0, 10, 20, 30, 40 and 50 minutes, respectively: P>0.01, P>0.01, P<0.01, P<0.01, P<0.01.

For both the coated and non-coated, groups, immersion in neither coffee nor red wine for up to 50 minutes caused a significant decrease in b* at any time point (P>0.05 for all time points; two-way ANOVA.)

Teething Lightening/Brightening by Whitening Pen of Invention

The effects of a whitening gel of the invention (Whitening Composition in a Gel Carrier) were compared to a prior art carbamide peroxide whitening gel (AURAGLOW® Teeth Whitening Pen, 35% carbamide peroxide available from AuraGlow, LLC, Stamford, Conn.), both administered by a pen applicator.

Twenty extracted teeth that appeared healthy to the naked eye and after inspection under a light microscope (10×) were bisected. Standardized photography and colorimetry measurements were taken at baseline. One-half of each tooth was coated two times with the whitening gel of the present invention; the remaining half of each tooth was likewise coated two times with the prior art carbamide peroxide gel of the present invention. A first coat was applied by a pen applicator and allowed to dry for about 30 seconds; a second coat was then applied.

Based on two-way ANOVA, statistically significant increases in L* values—indicative of teeth lightening—were observed in a time-dependent manner for both the whitening gel pen of the invention and the prior art carbamide peroxide whitening peroxide pen: treatment effect: P<0.001; time effect: P<0.0001). The whitening gel of the present invention lightened the tooth more quickly, with the first significant increase in L* values after 30 minutes of exposure (P<0.05, P<0.05, P<0.01, P<0.001, P<0.001 for progressive time points vs baseline, one way ANOVA followed by Tukey post-test). The carbamide peroxide gel produced a significant increase in L* values at 60 minutes (P>0.05, P<0.05, P<0.01, P<0.001, P<0.001 for progressive time points vs baseline, one way ANOVA followed by Tukey post-test).

Case Study

A 65-year-old female patient presents with severe gum recession and enamel erosion, causing root as well as tooth sensitivity. The patient has badly-stained teeth attributable to consumption of coffee (about 2-3 cups per day). Consequently, the patient experiences short, sharp pain in response to thermal, tactile and/or chemical stimuli—collectively a condition known as dentinal hypersensitivity. See G R Holland et al., “Guidelines for the design and conduct of clinical trials on dentine hypersensitivity. J Clin. Periodontol. Vol, 24, pp. 808-13 (1997). Dentinal sensitivity is assessed clinically by a jet of air and with an exploratory probe on the exposed dentin, in a mesio-distal direction, examining all the teeth in the area in which the patient complained of pain. The patient has been unable to tolerate in-office or at-home whitening treatments, including peroxide-based mouthwashes and toothpastes. The extent of pain from the peroxide whitening treatment is described as “intolerable”. The patient is able to use the whitening strips of the present invention on a once-daily basis (thirty minutes per day) for twenty-eight consecutive days without experiencing sensitivity. Throughout the 28-day treatment period, the patient continues to consume coffee (2-3 cups/day). Surprisingly, a visible whitening of the patient's teeth of seven (7) shades on the VITA TOOTHGUIDE 3D MASTER® tooth shade grading system is observed.

1. A method of visibly whitening tooth enamel or an outer surface of a dental restoration comprising the step of administering to the teeth an oral care product comprised of a tooth whitening complex consisting essentially of (a) a sea salt and (b) at least two essential oils selected from the group consisting of (i) lemon peel oil; (ii) coconut oil; and (iii) sage oil.

2. The method of claim 1 wherein the sea salt is Dead Sea salt, which is present at a concentration of from about 0.5% to about 4.0%.

3. The method of claim 2 wherein the oral care product is (a) a whitening dentifrice or (b) a whitening mouthwash, and the oral care product is further comprised of xylitol and aloe vera leaf juice.

4. The method of claim 3 wherein the oral care product is further comprised of at least one essential oil selected from the group of peppermint oil, wintergreen oil, and spearmint oil.

5. The method of claim 2 wherein the oral care product is (a) a whitening strip that is conformable to the teeth surfaces and to the interstitial spaces between teeth, without permanent deformation thereof or (b) a whitening gel, and the oral care product is further comprised of at least one gelling agent selected from the group of Carbomer, carboxymethyl cellulose, carboxypropyl cellulose, poloxamer, carrageenan, magnesium aluminum silicate, carboxyvinyl polymers, and natural gums such as gum karaya, xanthan gum, guar gum, gum arabic, gum tragacanth, and mixtures thereof.

6. The method of claim 5 wherein the oral care product is a whitening strip or a whitening gel, and the at least one gelling agent is a Carbomer and carboxymethyl cellulose, and the oral care product is further comprised of glycerol that is not petroleum derived.

7. The method of claim 3 wherein the oral care product is administered by a consumer for at least 30 seconds at least one time daily in the evening prior to going to sleep.

8. The method of claim 3 wherein the oral care product is administered for at least 30 seconds at least two times daily, a first time in the evening prior to going to sleep, and a second time in the morning after breakfast.

9. The method of claim 8 wherein tooth enamel or an outer surface of a dental restoration is visibly whitened by at least three shades as measured using the VITA TOOTHGUIDE 3D MASTER® tooth shade grading system.

10. The method of claim 6 wherein the oral care product is administered once daily for a period of time from about 15 minutes to about 30 minutes.

11. The method of claim 10 wherein the consumer abstains from eating or drinking for a period of at least about 30 minutes after administering the oral care product.

12. The method of claim 11 wherein tooth enamel or an outer surface of a dental restoration is visibly whitened by at least three shades as measured using the VITA TOOTHGUIDE 3D MASTER® tooth shade grading system.

13. A method of reducing staining or discoloration of teeth caused by (a) ingesting a consumable chromogenic substance or (b) smoking a cigarette wherein prior to ingesting a consumable chromogenic substance or smoking a cigarette, a person applies a whitening gel to the outer surface of the teeth in an amount sufficient to coat the teeth, and the whitening gel is comprised of a tooth whitening complex consisting essentially of (a) a sea salt and (b) at least two essential oils selected from the group consisting of (i) lemon peel oil; (ii) coconut oil; and (iii) sage oil.

14. The method of claim 13 wherein the person applies two coats of the whitening gel to each dental arch, wherein each coat is from about 0.25 ml to about 0.5 ml.

15. The method of claim 13 wherein the consumable chromogenic substance is coffee, tea, red wine, berries, soy sauce, curry, or smokeless tobacco.
